(19) United States
(12) Patent Application Publication
(10) Pub. No.: US 2015/0018925 A1
(43) Pub. Date: Jan. 15, 2015

(54) STIMULATION DEVICE

(71) Applicant: Cerbomed GmbH, Erlangen (DE)

(72) Inventors: Thomas ZSCHAECK, NUERNBERG (DE); Andreas HARTLEP, HOLZKIRCHEN (DE)

(21) Appl. No.: 14/327,057

(22) Filed: Jul. 9, 2014

(30) Foreign Application Priority Data
Jul. 10, 2013 (DE) 10 2013 011 530.2

(51) Int. Cl. A61N 1/04

(52) U.S. Cl. CPC A61N 1/0492
USPC 607/139

(57) ABSTRACT

A stimulation device for transcutaneous electric stimulation stimulus onto the surface of a section of the human ear comprises a holding element attached at or in the ear and at least two electrodes arranged in or at an electrode carrier, wherein the at least two electrodes are separated from another at the electrode carrier by an electrically isolating section. To obtain a good electrical conductibility of the electrodes at high ear comfort, the at least two electrodes are covered by a casing element, the casing element comprises at least two cover elements, wherein each of the at least two cover elements comprises a retaining region for an electrode; the material of the cover elements is electrically conductive or can be made electrically conductive; the casing element comprises at least one isolation section made of electrically isolating material; the isolating section connects at least two cover elements with another.

### CROSS-REFERENCE TO RELATED APPLICATIONS

This application claims the priority of DE 10 2013 011 530.2 filed Jul. 10, 2013, which is incorporated by reference herein.

### SUMMARY OF THE INVENTION

The invention relates to a stimulation device for the application of a transcutaneous electric stimulation stimulus onto the surface of a section of the human ear, which comprises a holding element which can be attached at or in the ear as well as at least two electrodes which are arranged in or at an electrode carrier, wherein the at least two electrodes are separated from another at the electrode carrier by an electrically isolating section.

A stimulation device of the generic kind is known from DE 10 2012 014 714 A1. DE 10 2010 015 277 A1 shows a similar solution.

It is generally known to take influence on the neurophysiological and neuroelectrical quality through invasive and non-invasive stimulation of the nerves and thereby on the function of the simulated nerves. Hereby different conditions of sickness can be treated. Numerous devices exist both for the invasive and the non-invasive stimulation.

The present invention is basing upon the method of the transcutaneous electrical stimulation of the nerves. At this method pulse currents of different current forms, amplitudes, pulse durations and frequencies are administered through the skin on different nerves and change their status parameter in an advantageous way.

A stimulation device of the kind mentioned above is known from DE 10 2010 054 165 B3. Here, an electrode arrangement is described which comprises a holding element which can be affixed at the ear. At the holding element an electrode carrier is arranged via an elastic section, which electrode carrier holds two electrodes. The electrode carrier consists of an electrical non-conductive plastic material and carries the two electrodes made of metal.

Although the pre-known stimulation device delivers already good treatment results certain drawbacks of the device have turned out in the practice. In this connection the contact quality of the electrodes has to be mentioned which is sometimes not yet optimal, because the skin areas which are reached by the electrodes are not sufficiently big. The increase of the contact pressure onto the skin surface is often no proper measure for the elimination of this difficulty because by doing so the skin of the patient is too much stressed.

Thus, it is an object of the invention, to further develop a stimulation device of the generic kind so that the mentioned drawbacks are eliminated. Thus, a further development has to be proposed so that the contact quality of the electrodes is improved while the skin surface of the patient isn't highly stressed and that a high wearing comfort is obtained.

### DETAILED DESCRIPTION OF THE INVENTION

The solution of this object by the invention is characterized in that the at least two electrodes are covered by a casing element, wherein the casing element comprises at least two cover elements, wherein each of the at least two cover elements comprises a retaining region for an electrode, especially a retaining chamber, wherein the material of the cover elements is electrically conductive or can be made electrically conductive, wherein the casing element comprises at least one isolation section made of electrically isolating material, wherein the isolating section connects at least two cover elements with another. Preferably, the two electrodes extend from the electrode carrier in two directions which are directed substantially away from another, wherein the casing element is designed substantially oblong.

The cover elements can be designed as hemispherical shells or as semi-ellipsoid shells, They correspond to the shape of the electrodes which they cover. An alternative provides that the at least two cover elements are designed hollow-cylindrical.

The isolation section comprises preferably a substantially cylindrical shape. The isolation section can thereby comprise a constriction in its middle region. Furthermore, the casing element can comprise an aperture for the passage of the electrode carrier.

According to an alternative embodiment it can be provided that the isolation section comprises a substantially rod-shaped form, wherein the axial ends of the isolation section are fixed at a circumferential location of the cover elements.

Preferably, the cover elements comprise of an open-pored porous material, wherein especially a sponge-like material is preferred. This material is preferably stretchable, so that it can lay elastically around the electrode which is covered by the cover element.

In this case it is preferred that the material of the cover elements is impregnated with a liquid or with a gel. This is the best mode for the above mentioned measure to make the material of the cover elements electrically conductive. Accordingly, the material of the cover elements is generally in fact open-porous but not electrically conductive. When bringing in a liquid or a gel (i.e. an electrolyte) the electrical conductivity is established.

The cover elements can also comprise of a closed-porous material, which is preferably soft and/or flexible. The electrical conductivity can then be established by other measures. An example for this is that carbon or metallic particles are mixed into the basic material for the cover elements what leads to the electrical conductivity of the produced cover elements,

The casing element is preferably designed as a one-piece part.

Preferably, the electrodes are metal electrodes, especially titan electrodes. They have preferably the shape of a spherical segment or of a section of an ellipsoid. The electrode carrier has, especially designed as electrode head, preferably at least one stimulation electrode and at least one reference electrode.

The parts of the stimulation device consist preferably—as far as skin contact is given—of a soft material, wherein specifically an elastomer material is considered, especially silicone or a material which contains silicone.

It is beneficially that a good contact quality of the electrodes during resting on the skin is given by the proposed embodiment of the stimulation device without requiring a high contact pressure of the electrodes on the skin of the patient.

### BRIEF DESCRIPTION OF THE DRAWINGS

In the drawing embodiments of the invention are depicted.

FIG. 1 shows the view of an ear with a stimulation device which is inserted into the ear (here still without casing element),

FIG. 2 shows the perspective view of a part of the stimulation device with electrode carrier and two electrodes, wherein a casing element is mounted at the electrode carrier,

FIG. 3 shows in exploded view the stimulation device according to FIG. 2, wherein the casing element is shown remote from the electrode carrier,

FIG. 4 shows a section through the casing element according to FIG. 2 and FIG. 3 respectively and

FIG. 5 shows in perspective view a casing element according to an alternative embodiment of the invention.

### DETAILED DESCRIPTION OF THE DRAWINGS

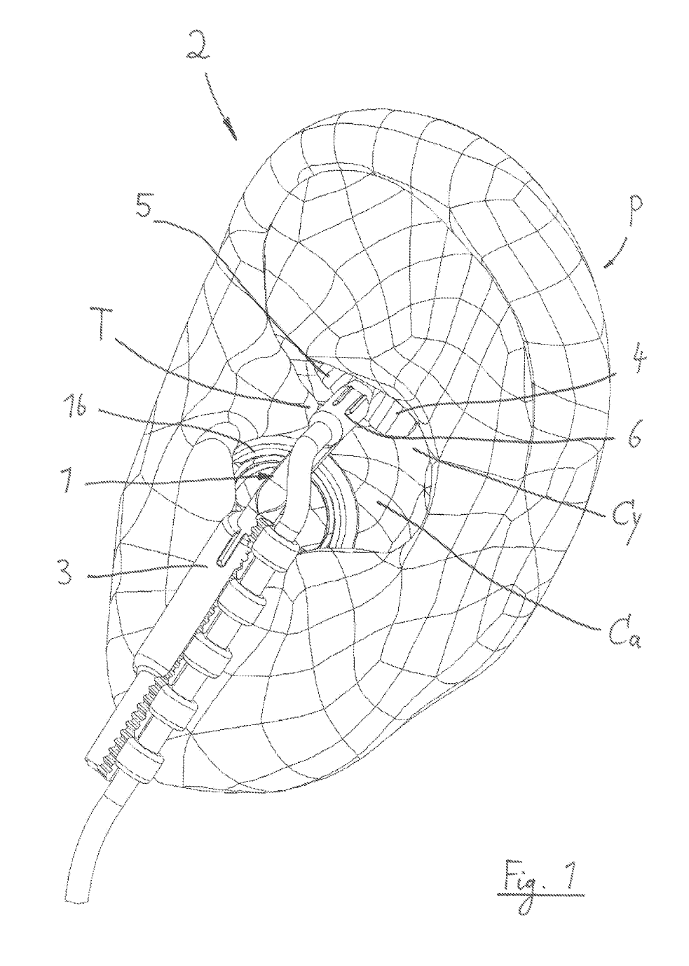

In FIG. 1 an ear 2 of a human is shown in which a stimulation device 1 is inserted to carry out a transcutaneous electrical stimulation. By doing so preferably the vagus nerve is subjected to an electrical stimulation to treat different diseases and to obtain effects respectively. Accordingly, with the stimulation device 1 a concrete transcutaneous electrical nerve stimulation can be carried out onto a surface area of the ear, For doing so the electrode arrangement comprises a stimulation electrode and a reference electrode (see below) between which an electrical potential is created; the means which are required for doing so are well known in the state of the art so that they need not be further described here. Exemplary, DE 10 2005 003 735 B4 of the applicant should be mentioned to which explicitly reference is made. The casing element (see below) is for sake of clarification not yet shown in FIG. 1.

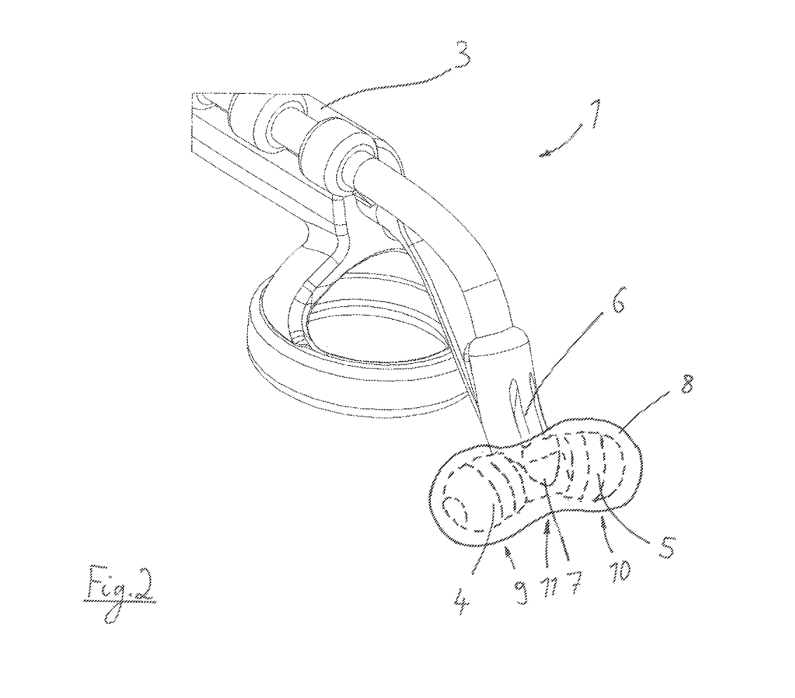

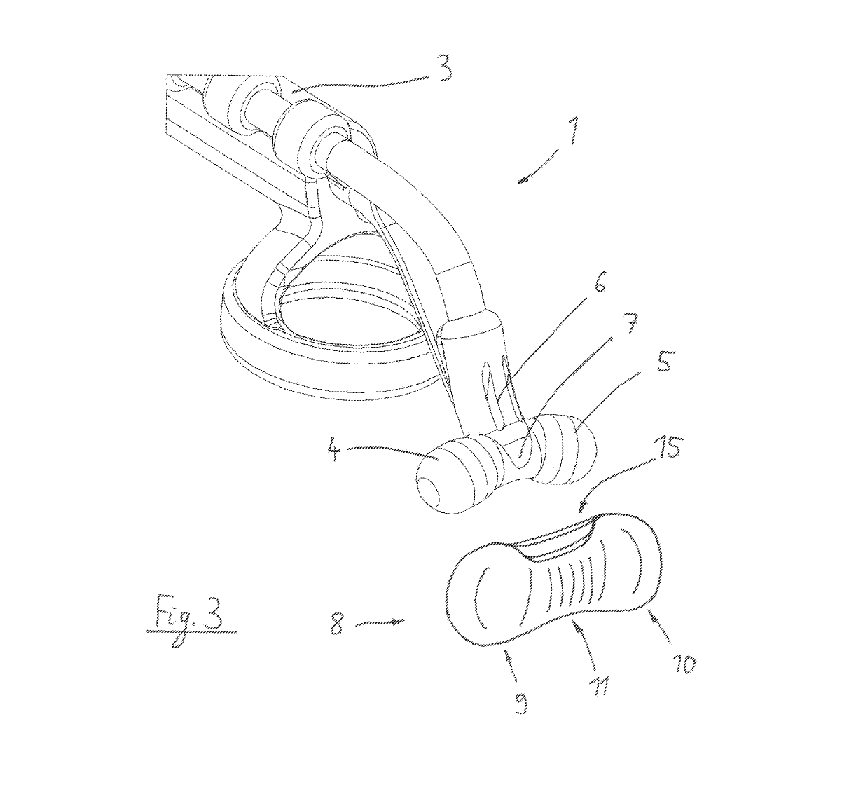

The stimulation device has—as it can be seen furthermore in FIGS. 2 and 3 a holding element 3 which carries an electrode carrier 6. In the electrode carrier 6 two electrodes 4 and 5 are arranged, wherein the electrode 4 is a stimulation electrode and the electrode 5 is a reference electrode. The two electrodes 4, 5 are separated from another by an electrical isolating section 7 of the electrode carrier 6 to create a difference of potential between the electrodes 4, 5, With respect to the design of the stimulation device reference is made explicitly to DE 10 2010 054 165 B3 of the application where this stimulation device is described in detail.

In FIG. 1 the Pinna P of the ear 2 can be seen. The electrode carrier 6 is located in the region of the Cymba conchae Cy; however the stimulation device 1 with a ring-shaped resting part rest in the region of the Cavum conchae Ca. Accordingly, the ring-shaped resting part 16 is arranged below the tragus T.

It is essential that the two electrodes 4 and 5 are covered by a casing element 8 as is can be seen in FIGS. 2, 3 and 4. The casing element 8 comprises in the present embodiment two cover elements 9 and 10, wherein each cover element has a retaining chamber 12 and 13 respectively for one electrode 4 and 5. Here, the material of the two cover elements 9 and 10 is made electrically conductive. This was carried out here in such a manner that an open-porous base material for the cover elements 9 and 10 was chosen, wherein otherwise not measures was taken to make the material electrically conductive which material consists of plastic. Then, the cover elements 9, 10 were impregnated with a gel for establishing said conductivity which gel soaked into the open-porous structure of the material of the cover element 9, 10 and so established the desired and required electrical conductivity,

Furthermore, the casing element 8 comprises an isolation section 11 made from electrical isolating material; this isolating section 11 connects the two cover elements 9 and 10 with another and isolated so also the difference of potential which exists between the electrodes 5, 6.

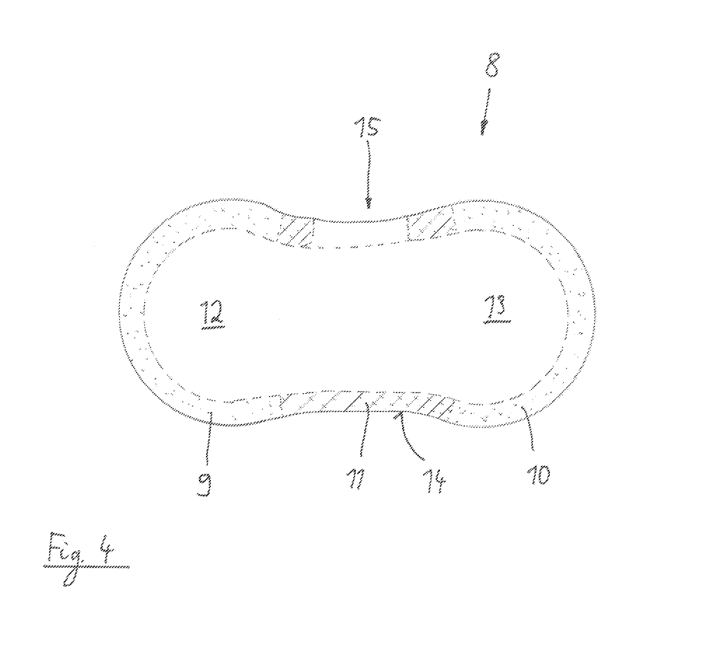

As can be seen best from FIG. 4, the two cover elements 9, 10 have respective retaining chambers 12 and 13 which fit to the form of the electrodes 9, 10. Thereby, the casing element 8 and namely the cover elements 9, 10 are produced a bit smaller as it corresponds to the form of the electrodes 4, 5. This allows that the casing element 8 is arranged with a small pre-load during mounting onto the electrode carrier 6 including electrodes 4, 5 so that the surface of the electrodes 4, 5 is pressed against the inner surface of the cover elements 9, 10. Furthermore, a good fit of the casing element 8 on the electrode carrier 6 is promoted by a constriction 14 of the isolation section 11 which has a substantial hollow-cylindrical shape apart from that,

For the insertion of the electrode carrier with electrodes the casing element 8 has an aperture 15. The elasticity of the material of the casing element 8 allows that the casing element lies against the electrode carrier 6 and the electrodes 4, 5 at a relatively small size of the aperture 15.

Presently, the two electrodes 4 and 5 extend from the electrode carrier 6 in two directions which are directed away from another. Accordingly, the casing element 8—as can be best seen in FIG. 4—is designed as a longitudinal extending part.

The casing element 8 can be made—as can also be seen from FIG. 4—as a one-piece part, wherein different materials can be employed, at the one hand for the cover elements 9. 10 and on the other hand for the isolation section 11.

It would also be possible that the casing element is fixed by means of further fixation means at the electrode carrier. Hereby, a rubber band can be contemplated which tensions the casing element along the circumference of the aperture 15 onto the electrode carrier 6.

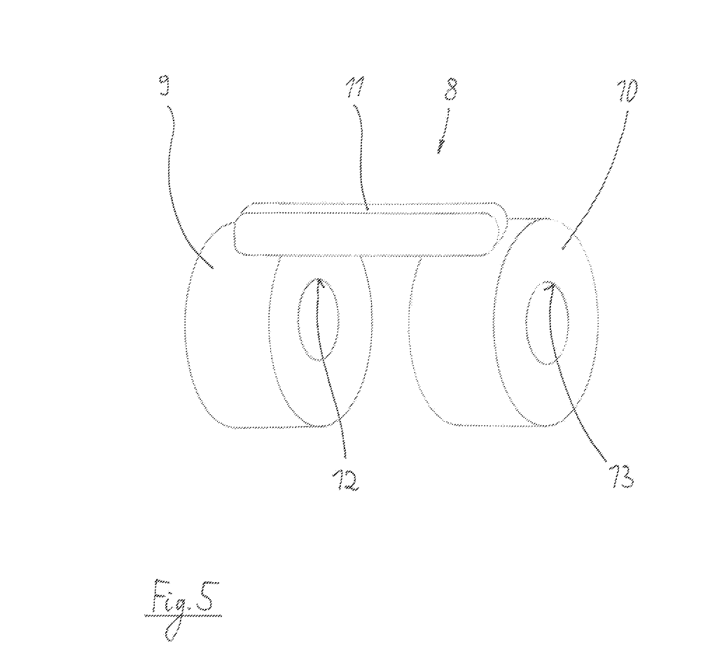

In FIG. 5 an alternative design of the casing element 8 can be seen which is here performed as a kind of cleading for the electrodes 4, 5. Again, the two cover elements 9 and 10 are provided which are designed hollow-cylindrical here. The casing element 8 is produced by applying (e, g, gluing or welding) of the isolation section 11 which is rod-shaped here. Be doing so the two cover elements 9, 10 are held at a defined distance electrically isolated against another. The electrodes 4, 5 are inserted into the cylindrical retaining regions 12 and 13. Again it applies that the cover element 9, 10 must be electrically conductive, while the isolation section 11 must be electrically isolating.

A plurality of casing elements 8 can also be stored in an applicator. The applicator can have a respective number of storing pockets which are sealed by a plastic foil impermeably. Each one casing element 8 can be placed in a respective storing pocket. If a new casing element 8 is required the storing pocket is opened and the casing element it taken off.

Preferably, it is thereby provided that a suitable electrolyte is already brought into the impermeably sealed pocket, so that the cover elements of the casing element are kept in a desired, i. e. electrically conductive state in which the casing element 8 is kept under optimal conditions for the transcutaneous stimulation till its use.

### LIST OF REFERENCES

1 Stimulation device
2 Ear
3 Holding element
4 Electrode (stimulation electrode)
5 Electrode (reference electrode)
6 Electrode carrier
7 Electrically isolating section
8 Casing element (envelope)
9 Cover element
10 Cover element
11 Isolation section
12 Retaining region (retaining chamber)
13 Retaining region (retaining chamber)
14 Constriction
15 Aperture
16 Ring-shaped resting part
Ca Cavum conchae
Cy Cymba conchae
T Tragus
P Pinna

1. Stimulation device for the application of a transcutaneous electric stimulation stimulus onto the surface of a section of the human ear, which comprises a holding element which can be attached at or in the ear as well as at least two electrodes which are arranged in or at an electrode carrier, wherein the at least two electrodes are separated from another at the electrode carrier by an electrically isolating section,
wherein
the at least two electrodes are covered by a casing element, wherein the casing element comprises at least two cover elements, wherein each of the at least two cover elements comprises a retaining region for an electrode, wherein the material of the cover elements is electrically conductive or can be made electrically conductive, wherein the casing element comprises at least one isolation section made of electrically isolating material, wherein the isolating section connects at least two cover elements with another.

2. Stimulation device according to claim 1, characterized in that the at least two electrodes extend from the electrode carrier in two directions which are directed substantially away from another, wherein the casing element is designed substantially oblong.

3. Stimulation device according to claim 1, characterized in that the at least two cover elements are designed as hemispherical shells or as semi-ellipsoid shells.

4. Stimulation device according to claim 1, characterized in that the at least two cover elements are designed hollow-cylindrical.

5. Stimulation device according to claim 1, characterized in that the isolation section comprises a substantially cylindrical shape.

6. Stimulation device according to claim 5, characterized in that the isolation section comprises a constriction in its middle region.

7. Stimulation device according to claim 1, characterized in that the isolation section comprises a substantially rod-shaped form, wherein the axial ends of the isolation section are fixed at a circumferential location of the cover elements.

8. Stimulation device according to claim 1, characterized in that the casing element comprises an aperture for the passage of the electrode carrier,

9. Stimulation device according to claim 1, characterized in that the cover elements comprise of an open-pored porous material, especially of a sponge-like material, which is preferably soft and/or flexible.

10. Stimulation device according to claim 9, characterized in that the material of the cover elements is impregnated with a liquid or with a gel.

11. Stimulation device according to claim 1, characterized in that the cover elements comprise of a closed-porous material, which is preferably soft and/or flexible.

12. Stimulation device according to claim 1, characterized in that the casing element is designed as a one-piece part.
